(19) United States
(12) Patent Application Publication
(10) Pub. No.: US 2004/0265281 A1
(43) Pub. Date: Dec. 30, 2004

(54) System capable of treating and defining various diseases using stem cells

(76) Inventors: Denis Rodgerson, Malibu, CA (US); Thomas Hirose, Huntington Beach, CA (US); Rubio Punzalan, Pasadena, CA (US)

Correspondence Address: Sung I. Oh, Esq., Squire, Sanders & Dempsey, L.L.P., 14th Floor, 801 S. Figueroa Street, Los Angeles, CA, 90017, US

(21) Appl. No.: 10/819,398

(22) Filed: Apr. 7, 2004

Related U.S. Application Data
(60) Provisional application No. 60/461,398, filed on Apr. 7, 2003.
(60) Provisional application No. 60/461,543, filed on Apr. 7, 2003.
(60) Provisional application No. 60/461,584, filed on Apr. 7, 2003.
(60) Provisional application No. 60/461,614, filed on Apr. 7, 2003.

(51) Int. Cl. A61K 45/00; C12Q 1/68; G01N 1/30; G01N 33/48; C12N 5/08

(52) U.S. Cl. 424/93.7; 435/40.5; 435/366

(57) ABSTRACT

A system provides individualized “baseline” or internal “control” for an individual by collecting cells from the individual at an earlier time point prior to the onset of disease, which can be used as a standard for normalcy and a benchmark or reference for measuring the progression or development of any process the cells undergo. This allows study of biophysical, biochemical, architectural, morphological, functional, or physiological differences between cells collected from an individual at one time to cells collected from the same individual at a later point in time. The comparison between cells collected at different times allows for the study of relative responses of these cells when treated with a variety of chemicals. With this system disease markers can be determined that can aid in diagnosis and discovery of therapeutics for the disease of interest. As a result, this system may provide a basis for the production of patient-specific, as opposed to disease-specific drugs.

### RELATED APPLICATIONS

This application claims priority to four provisional applications having application numbers 60/461,398, 60/461,543, 60/461,584 and 60/461,614, all filed Apr. 7, 2003, which are incorporated by reference.

### BACKGROUND OF THE INVENTION

1. Field of the Invention

The present invention relates to diagnostic and therapeutic uses of autologous cells collected from an individual at different points in time by comparing and analyzing the cellular integrity of the cells. A method in accordance with this invention is applicable for the analysis of the possible causes of different kind of diseases. More specifically, the invention provides methods of using adult stem cells collected from the person prior to diseased state, using epigenetic information of these cells as reference points and compare with various epigenetic information collected at the time of disease or at multiple intervals prior to diseased state. The method in accordance with this invention with the stem cells collected prior to diseased state can also be re-infused to the same person during or after the development of various diseases including cancers, infectious disease, neutropenia and aplastic anemia. As such, in one embodiment, the invention also provides methods of using adult stem cells collected from the person prior to diseased state and re-infused to the same person post during or after the development of cancer.

2. Description of the Related Art

Current studies of disease start out of necessity—with already diseased cells. Comparison of diseased cells from a large population of individuals to locate the common gene(s) that are turned on or off or otherwise modified is equivalent to looking for a needle in a haystack, or sorting by brute force. One of the reasons for the identified difficulty may be that the genes from a population of different individuals may differ due to harmless polymorphism or demographical factors such as gender or race in the population studied.

Stem cell transplantation has been used either by itself (e.g. for congenital diseases) or in conjunction with other treatments such as chemotherapy for treating cancer carrier persons. However, most of these stem cell transplants either use stem cells from matched donors (allogeneic) or collecting stem cells from the persons right before their treatment (autologous). In allogeneic transplantations, there are number of drawbacks including immune rejections and graft-versus-host-diseases. In addition, allogeneic transplantation is more expensive than autologous transplantation. Infectious agents such as bacteria, virus, fungi, parasite, prion protein, etc., may be in the peripheral blood during the diseased state (sepsis). As a result the stem cells obtained from the septic patients during or after the diseased state may be contaminated with infectious agents such as bacteria, virus, fungi, parasite, prion protein, etc. This may lead to treatment failure due to re-infusion of stem cells contaminated with the infectious agents. This invention provides a method and facility to collect, process, and store individual's healthy stem cells for their future treatments against infectious diseases, autoimmune diseases, cancers or other conditions.

Tumor cells may circulate in the blood during or after the diseased state. As a result the stem cells obtained from the cancer patients during or after the diseased state may be contaminated with tumor cells. This may lead to treatment failure due to re-infusion of stem cells contaminated with tumor cells. Stem cells collected from umbilical cords may be used for some cancer treatments, but the low cell dose, immaturity and incomplete complement of cells limits the immediate use of these stem cells for cancer treatments. This invention also addresses various problems associated with the prior arts by providing a method and facility to collect, process, and store individual healthy stem cells for their future treatments against tumor in the event that they develop cancer.

### SUMMARY OF INVENTION

This invention provides individualized “baseline” or internal “control” for an individual by collecting cells from the individual at an earlier time, which can be used as a standard for normalcy and a benchmark or reference for measuring the progression or development of any process the cells undergo. This allows study of biophysical, biochemical, architectural, morphological, functional, or physiological differences between cells collected from an individual at one time to cells collected from the same individual at a later point in time. The comparison between cells collected at different times allows for the study of relative responses of these cells when treated with a variety of chemicals. With this invention disease markers can be determined that can aid in diagnosis and discovery of therapeutics for the disease of interest. As a result, this invention may provide a basis for the production of patient-specific, as opposed to disease-specific drugs.

### DETAILED DESCRIPTION OF THE PREFERRED EMBODIMENTS

The present invention applies to all animals, in particular vertebrates. Examples of such vertebrates are mammals. An example of such a mammal is a human such as a human baby, child, or adult. For ease of discussion in this patent application, we use a human being (identified as a person or a patient) as a non-limiting example of such an animal. Also, for ease of discussion, the pronoun “he” is used. It is to be understood that the term “he” includes “he” and/or “she”. Following longstanding law convention, the terms “a” and “an” as used herein, including the claims, are understood to mean “one” or “more”.

The present invention presents methods for using autologous-stem cell transplants, such as those from peripheral blood, and bone marrow from post-birth human (including neonate and adult), for the treatment of infectious disease, neutropenia and aplastic anemia, cancers or other conditions. In a non-limiting example of the invention, the diseases treated are CMV sepsis, tuberculosis, cryptosporidium, pneumocystis camii, syphilis, anthrax, Yersinia pestis, amaebe, trypanosome Cruzii, spongiform encephalitis (new variant Creutzfeldt-Jacob disease), malaria, and schistosomiasis and acquired immunodeficiency syndrome (AIDS). The invention has an advantage over umbilical cord blood transplants since for most children and adult, their umbilical cord blood at birth is no longer available.

To provide a baseline for an individual, cells are harvested from the individual. Such cells may be in a “pre-disease” stage. The term “pre-disease” generally means that the state, in which the individual is healthy, before the individual has developed, manifested, become symptomatic, or are diagnosed with a disease. The term includes both the absolute term of “healthy” or “no disease” and the relative term of a gradation in the disease progression such as “healthier than” or “less diseased”.

These cells are collected and preserved in such environment that their cellular integrity is substantially preserved. The term “cellular integrity” generally includes but not limited to biophysical, biochemical, architectural, morphological, functional, and physiological aspect of cells. One example of preserving cellular integrity is cryopreservation.

In a later point in time, cells are collected from the same individual. The individual may still be at the “pre-disease” stage or may have developed, manifested, become symptomatic, or been diagnosed with a disease. By way of example, such individual may be harvesting “initial-disease” or “post-disease” cells. The term “initial-disease” generally denotes the state at which the individual is developing, manifesting, becoming symptomatic, or has been diagnosed with a disease at its early stage. The term “post-disease” generally denotes the state at or after which the person has developed, manifested, become symptomatic, or diagnosed with a disease, or the disease has become detectable or been detected.

The cells collected at the earlier point in time may then be retrieved from storage. In the example in which the cells are cryopreserved, the cells may be thawed. In one embodiment, the invention also provides a novel method of identifying the biological material for positive identification for cryopreservation. These cells function as the control which sets a standard for normalcy.

The cells collected at the later point in time may then be compared to the cells collected at the earlier point in time. By way of example, the “initial-disease” cells may then be compared to the “pre-disease” cells; the “initial-disease” cells may be compared to “post-disease” cells; and the “post-disease” cells may be compared to “pre-disease” cells.

A non-limiting example of comparison between cells collected at different times include molecular differences such as: differences in their DNA, RNA, or protein expression, regulation, repression, transcription, or translation.

By way of example, a progression of a disease may be mapped by comparing the differences between the cells collected as the individual deteriorates with the disease to the pre-disease cells. It may be observed that the less a protein of interest is expressed, the more fragmented or changed the protein population becomes as compared to the protein secreted by the pre-disease cells.

By way of example, such progress may be observed on gel electrophoresis when the lysate from the pre-disease cell is compared to that from the initial-disease cells or the post-disease cells. Certain bands of protein from the lane for the pre-disease cells may not be reflected in the lane for the initial-disease cells or the post-disease cells, indicating absence of such protein in the initial-disease cells or the post-disease cells which may account for the disease. It may be that the lane for the initial-disease cells or the post-disease cell shows new protein bands of smaller molecular weight, which may indicate the fragmentation of a normal protein or expression of new proteins of smaller molecular weight.

In another embodiment of the invention, the method which compares the differences between the pre-disease and initial-disease cells or post-disease cells from an individual can be used to monitor the progression of disease. For ease of discussion, the following uses cancer/tumor as a non-limiting example of a disease.

Physicians and scientists can assess and catalog the differences in gene expression between the pre-disease and initial-disease or post-disease cells by analyzing the changes in their patterns of gene expression. For example, physicians and scientists can assess and catalog the differences in gene expression between the pre-disease cells by analyzing the changes in its patterns of gene expression compared with solid tumor from the individual. Thus, cancer can be diagnosed at earlier stages before the patient is symptomatic. For example, if the patient has a family history of predisposition to cancer, such as breast cancer, she can be tested at intervals of time to observe any changes in her gene expression.

This invention can also be used to monitor the efficacy of treatment of a disease. For some treatments with known side effects, the invention is employed to “fine tune” the treatment regimen. A dosage is established that causes a change in genetic expression patterns consistent with the genetic expression of the pre-disease cell. Expression patterns associated with undesirable side effects are avoided. This approach may be more sensitive and rapid than waiting for the patient to show inadequate improvement, or to manifest side effects, before altering the course of treatment. Instead of, or in addition to reviewing the above genetic differences, the physician may also use proteomic techniques to assess and catalog the differences in protein expression between the pre-disease healthy tissues by analyzing changes in its patterns of protein expression compared with solid tumor from the patient, to achieve the same diagnostic and therapeutic ends.

Another non-limiting example of comparison between cells collected at different times include differences in the nature of the genetic materials, the quantitative and qualitative nature of the protein expressed such as changes in protein structure, or the amount or lack of genetic materials or expression, or protein secretion. By way of example, one skilled in the art may assay for differences in the nature of the genetic materials in the cells to detect, identify, or locate any mutation in the genome and the genetic transcription.

Further non-limiting example of comparison between cells collected at different times lies in which the cells are cultured and grown in controlled environment in vitro. Various chemicals, drugs, hormones, or external factors may be applied to the respective cells to determine any differences in their behavioral responses. By way of example, because the present invention readily avails the pre-disease cells, the pre-disease cells and the cells collected at a later time can be tested side-by-side for drug treatment. The pre-disease cells and its behavior may serve as a control to which a person of ordinary skill in the art can resume the initial-disease or post-disease cells. If need be, the pre-disease cells may be cloned to provide greater quantity of cells for necessary purposes.

The differences between the cells collected at different times may be compared and analyzed at different levels by comparing one or more of their following features: their DNA, RNA, RNA transcriptions (mRNA), proteomic, immunologic differences, and/or their different reaction to drugs, chemicals, environmental or other exogenous factors. Various known techniques may be used to accomplish the foregoing comparison and analysis, many of which form the basis of clinical diagnostic assays.

These techniques may include:

(1) nucleic acid (DNA or RNA) hybridization analysis, to determine the changes in the DNA which makes up the person's genome; and to determine changes in the expression and the resulting mRNA; (2) restriction enzyme analysis; (3) genetic sequence analysis; (4) separation and purification of nucleic acids and proteins; and (5) in vitro drug, chemical, environmental testing.

All the analysis from items (1) to (4) above are described in detail in Sambrook et al., Molecular Cloning: A Laboratory Manual, Second Ed. (Cold Spring Harbor Laboratory Press, Cold Spring Harbor, N.Y., 1989), which is hereby incorporated by reference. For detailed discussion of various prior art analytical methods, see for example, U.S. patent application No. 2002/0,119,565 A1, of Clarke, et al., “Isolation and Use of Solid Tumor Stem Cells”, published Aug. 29, 2002; U.S. patent application No. 2002/0,012,932 A1 of Wang, “Microchip Arrays of Regulatory Genes”, publ. Jan. 31, 2002; and U.S. patent application No. 2003/0,017,508 A1 of Charych, “Microarrays on Mirrored Substrates for Performing Proteomic Analyses”, publ. Jan. 23, 2003, which are all incorporated by reference.

### 1. Infectious Diseases and Stem Cells

Mechanisms that cause normal tissues to become infected involve an enormously complex process. Indeed, complexity in producing sepsis occurs at each of many hierarchical levels. Even at the genetic level, certain genetic states are more susceptible to infection. Infection is also the outcome of altered mechanisms occurring at other levels involving RNA, proteins, intracellular pathways, intercellular interactions, tissues, organs, etc. Since events occurring at one hierarchical level feed into and modify mechanisms at other levels, infection is a dynamic process that is more complicated than a simple summation of the parts. An important consideration is that some infection may originate from defective stem cells.

There is no prior art for the use of stem cells in the treatment of sepsis or infectious disease. If stem cells were collected at the time of sepsis, they may be contaminated with infectious agents such as bacteria, virus, fungi, parasite, prion protein, etc. Thus the collection of peripheral blood stem cells during individual's healthy state (pre-disease) coupled with infusion at the time of sepsis or infectious disease may lead to significantly improved mobility and mortality.

Applicants recognize the benefits of stem cell transfusion which would ultimately result in increased survival rates, while at the same time providing a low-cost, clinically effective method for treating patients with infectious disease, neutropenia and aplastic anemia using their own stem cells collected while the patients are in healthy state.

### 2. Applicants' Hypotheses

Without wishing to be bound by the hypotheses postulated in this application, applicants made the following hypotheses. Each hypothesis may or may not relate to the other hypotheses. The efficacy of the invention in practice is obviously not bound by the correctness of the hypotheses.

As a first hypothesis, applicants believe that many infections are systemic in nature at the time of diagnosis and widely distributed throughout the body. That is, applicants believe that by the time a patient has been diagnosed with many types of infection, e.g., CMV sepsis, tuberculosis, cryptosporidium, Pneumocystis carnii, syphilis, anthrax, Yersinia pestis, amoebae, Trypanosome cruzii, spongiform encephalitis(new variant CJO), malaria, schitosomiasis and acquired immunodeficiency syndrome (AIDS), there can already exist infectious agents such as bacteria, virus, fungi, parasite, prion protein, etc in other parts of the patient (e.g. blood) besides the perceived affected area. In fact, the infectious may have already spread or migrated throughout the patient's body, for example, as the infection agents are being carried by the patient's circulating blood or lymphoid system. This hypothesis accounts for contamination of stem cells by infection agents therefore if stem cells are collected after disease state, they would not be effective or contra indicated for treating infectious disease.

As a second hypothesis, applicants believe that in some instances, infectious agents such as bacteria, virus, fungi, parasite, prion protein, etc., are routinely circulating in low numbers in the human body. However, the human does not develop sepsis because his normal cells “self-regulate” the body by monitoring and eliminating the infectious agents before they proliferate uncontrollably and give rise to sepsis. Applicants further postulate that in some patients, their previously healthy cells become diseased because their diseased cells have partially or completely lost the ability to “fight infection” due to old age, and/or environmental assaults (exposure to radiation, carcinogens, or stress, etc.); and/or other factors as yet unknown. For example, the cell loses the ability to produce an enzyme or chemical necessary for the body's proper functioning resulting in shingles, abcesses, etc., or failure to generally sustain normal functioning of the body. Thus, transplantation of such already diseased (defective) cells harvested from these patients immediately prior to transplantation may not be helpful.

As a third hypothesis, the applicants believe that the stem cells collected from individuals at healthy state may be combined with infectious agents such as bacteria, virus, fungi, parasite, prion protein, etc., in the laboratory to program and increase the ability of the stem cells to combat the targeted infectious disease once infused back into the individuals. When programming stem cells in this manner, infectious agents may be either neutralized (non infectious) before combining with stem cells or the stem cells may neutralize (non infectious) the infectious agents prior to infusing to the patients.

### 3. The Invention

Thus, the present invention provides the following method:

(1) healthy cells are harvested from a person in the “pre-disease” stage. The term “pre-disease” means indicate the state in which the patient is healthy, or before the patient has developed, manifested, or been diagnosed with a disease,

(2) the pre-diseased cells are preserved and stored, e.g., by cryopreservation;

(3) in the event that the person later develops, manifests, or been diagnosed with a disease (“post-disease” state), he is infused with the above previously preserved pre-disease cells. The term “post-disease” denotes the state at or after which the person develops, has developed, manifests, has manifested, has been diagnosed with a disease, or his disease has become detectable or been detected.

It should be noted that the above discussion over “pre-disease” state (versus “post-disease” state) covers the absolute term of “healthy/no disease” (versus “not healthy/diseased”) and a relative term of a gradation in the disease progression (“healthier than” or “less diseased” than post-disease state). Since “pre-disease” can be defined by a time prior to a person being diagnosed with a disease, he could be healthy in an absolute term or he might already have the disease but only that it has not manifested itself, been diagnosed or detected. Even in the latter scenario, for such a “pre-disease” state, it is possible that the disease (infectious agent) may not be so widespread such that it has reached the cells collected or may be in a low amount that will not induce further infection upon re-injection even if the cells collected are diseased. They may be still retaining some functioning necessary to combat the disease. Thus, the term “healthy” cells covers both the absolute term that the cells are healthy, and the term that, relatively speaking, these collected cells are healthier than what the patient (in his “post-disease” state) currently have in his body.

### 4. Advantages of the Present Invention

The present invention has the following advantages:

(1) Since the method uses autologous transplant, there is no need to expand time, money and energy in “cleansing” the SC or donor marrow of potentially dangerous mature T cells (thought to be important for the development of graft-versus-host-disease. This is especially advantageous because such cleansing process may introduce harmful impurities into the bone marrow. Examples of cleansing process are: chemicals or monoclonal antibody (OKT3) that specifically recognizes and eliminate mature T cells.

In the case of patients with other diseases, the healthy harvested cells will not contain the disease vectors (e.g. CMV sepsis, tuberculosis, cryptosporidium, Pneumocystis carnii, syphilis, anthrax, Yersinia pestis, amaebe, Trypanosome cruzii, spongiform encephalitis (new variant CJD), malaria, schistosomiasis and acquired immunodeficiency syndrome (AIDS)), such as in the case of AIDS patients, the pre-disease cells will not contain the AIDS virus;

(2) Since the method uses autologous transplant, there is no need to laboriously locate SC or bone marrow from other donor and to conduct tests to ensure that the SC or bone marrow matches that of the recipient. Further, there is no fear that matching SC or bone marrow may not be found or that crucial time is lost to test and locate such matching SC or bone marrow such that the recipient may be in mortal danger or be deceased by the time a match is found;

(3) Since the method uses autologous transplant, there is no fear of graft-versus-host disease; or immune rejection.

(4) Since the method uses autologous transplant, there is no fear of transplant transmitted disease (e.g. HIV, CMV, hepatitis, syphilis etc.)

(5) Of particular importance is the fact that these autologous transplants are harvested prior to the development of disease (pre-disease stage) as compared to the post-disease stage. The pre-disease state autologous transplant has the following advantages over the post-disease state autologous transplant:

(I) The previously harvested pre-disease cells will be younger—Among the possible advantages associated with youth are: the cells will likely to be more resilient, more versatile, and would retain normal (or relative more normal) activities and a full range of (or broader range of) activities, and thus more well-equipped and more vigorous in combating a disease, as compared to the post-disease cells. Further, due to advance in age (e.g., in old age), certain population of cells may be depleted or no longer be available for harvesting at a later stage in a human's life. Also, certain functions or genes in older cells may be turned off, down-regulated, or lost, due to the natural aging process, aged related deterioration, mutation, or accumulated “wear-and-tear”, or environmental assaults over the years, etc. Further, older cells (post-disease versus pre-disease cells) may contain more mutations, defects, due to age or mistakes in the replication process, or environment assaults.

(II) The previously harvested (pre-disease) cells will be healthy cells (or healthier than the current cells existing in the patient) and can be expanded or programmed, or more readily programmable than the post-disease cells.

(III) The pre-disease population of the harvested cells will be healthy or will contain more healthier (or less diseased) cells than the post-disease population of cells, and thus the population of the pre-disease harvested cells will not be contaminated, be altered or contain infectious agents.

For example, in the case of infectious disease treatment, the present invention has the following advantages: the infused cells will not be contaminated with infectious agents such as bacteria, virus, fungi, parasite, prion protein, etc., (or will be less contaminated with infectious agents if the collection occurred after the disease has taken hold but before its diagnosis) as compared to the cells collected from a patient who has already developed infectious disease. Therefore, no laborious, time-consuming, inefficient methods (that may even inadvertently introduce undesirable chemicals) assays and screenings are required to cleanse the harvested cell population to remove the infectious agents from the pre-disease harvested cells. Further, the present invention eliminates or reduces the possibility of causing a relapse through infusion of multiple infectious agents.

(IV) The population of harvested cells may be more normal, complete in complement or more healthy in that a full range of normally occurring cells will be present relative to diseased cell population. For example, the peripheral blood SC collected from an AIDS patient will be deficient in T-Helper cells which are decimated by the AIDS virus; whereas these T-Helper cells would be found in the previously healthy population of cells.

(V) Furthermore, hematopoietic stem and progenitor cells can potentially be multiplied in culture, before or after cryopreservation, thus expanding the number of stem cells available for therapy. Thus, the population of healthy cells may be increased by expansion and infused into the patient to greatly boost his immunodefense in the number of cells available and that the cells are healthy.

(VI) Furthermore, processed hematopoietic stem cells may undergo immuno modulation or cellular adaptation inherent in the processing and cryopreservation technique leading to more effective immune functions.

### 5. An Embodiment of the Invention

Thus, in one embodiment of the invention: the stem cells of a neonate or an adult (“person”), while the child or adult is in a pre-disease state, are harvested and then preserved (such as cryopreservation). The harvesting (collection) process can be achieved using apheresis. There may be a need to infuse cell growth factors such as Granulocyte Colony Stimulating Factor 3-6 days prior to the collection. To preserve the stem cells collected for future used, cryopreservation can be used.

Later (and this may be years later), should the same person develop infectious disease, an immunodisease, cancer or undergoes therapy or is exposed to conditions which causes immunosuppression or infection or depletion of his immune cells, then the preserved stem cells or bone marrow are infused into the person to combat the disease. This can be achieved by intravenous infusion of the stem cell products. Similarly, the treatment protocol, and the criteria for determining the progress of the person and for adjusting the amount/dosage of cells to be infused can be achieved using standard transplantation practice.

Of course, the amount of stem cells collected should be sufficient for a major transplantation. If needed to, multiple collections should be done at an appropriate interval between collections (typically 3-7 days apart). However, as medicine advances, the preserved cells can be expanded and made to multiply or differentiate into the desired cell types before infusion into the person. If the person is deficient in certain subpopulation of cells, the subpopulation of cells from the preserved or expanded cells may be selected for in the future, and infused into the person. Furthermore, the harvested or expanded cells can be programmed by growing them in vitro with the person's diseased cells or tissues, or under stimulation by desired chemicals or cytokines before selecting for the desirable programmed cell and infusing them into the person. All these are conceivably achievable in the future.

In this embodiment, stem cells and bone marrow cells are chosen because they are versatile and because of their known use in cancer and immunodisease treatments and known methods for harvesting, preserving, expanding them. Their use in such treatments may be employed in this invention. The following describes this embodiment in further details.

### 5(a) Infectious Disease Treatment

For ease of discussion, the following use infectious diseases, such as CMV sepsis, tuberculosis, cryptosporidium, Pneumocystis carnii, syphilis, anthrax, Yersinia pestis, amoebae, Trypanosome cruzii, spongiform encephalitis, malaria, schistosomiasis and acquired immunodeficiency syndrome (AIDS), as a non-limiting example of infectious disease. The incidence of fatal infectious disease is the third highest after stroke and heart attack in the world. Infectious disease, neutropenia and aplastic anemia are difficult diseases to treat. Mini-dose of autologous stem cells may be used to augment a patient's immune system during the treatment of infectious diseases, autoimmune diseases, cancers and other diseases.

### 5(b) Methods For SC Collection, Selection, Preservation and Infusion

Conventional methods for collecting, cryopreserving, thawing, screening for and quantifying stem cells, and selecting for subpopulations of the stem cells, may be used.

Apheresis collection process from peripheral blood is the most common for collecting stem cells today. Hematopoietic cells can be isolated from suitable animal or human tissues including, for example, peripheral blood and bone marrow. Mononuclear cells, for example peripheral blood mononuclear cells (PBMCs) may be further isolated by methods such as density-gradient centrifugation. Sufficient quantity should be collected. If needed to, multiple collections should be considered to ensure enough doses for most demanding transplantation, typically about one (1) billion cells. The stem cells may be preserved by cryopreservation and later thawed for use, using standard transfusion procedures.

In an embodiment of the invention, all the stem cells collected can be cryogenically preserved, and used for hematopoietic reconstitution after thawing, in order to avoid cell losses associated with cell separation procedures. However, it is envisioned that cell separation procedures can be used if desired.

In one embodiment of the present invention for the primitive cell population to be further subdivided into isolated subpopulations of cells that are characterized by specific cell surface markers. The methods of the present invention may further include the separation of cell subpopulations by methods such as high-speed cell sorting, typically coupled with flow cytometry.

### 5(c) Infusion and Transplantation

Conventional standard transfusion methods (e.g. intravenous infusion) may be used for infusing the stem cells. Standard protocols for chemotherapy may be used followed by stem cells infusion for bone marrow reconstitution.

### 5(d) Confirmation That the Transplant is Working

Normal conventional practice should be observed to monitor the progress of the patient undergoing transplantation. However, the applicants' method should reduce the amount of potential complications resulting from immune rejection, graft-versus-host-diseases, the duration of engraftment during the treatment of infectious disease, neutropenia and aplastic anemia.

This invention also presents methods for using autologous stem cell transplants, such as those from peripheral blood, and bone marrow from post-birth human (including baby, child and adult), for the treatment of diseases. In a non-limiting example of the invention, the diseases treated are cancer and immunodiseases such as acquired immunodeficiency syndrome (AIDS). The invention has an advantage over umbilical cord blood transplants since for most children and adult, their umbilical cord blood at birth is no longer available.

### 1. Cancer and Stem Cells

Mechanisms that cause normal tissues to become malignant involve an enormously complex process. Indeed, complexity in carcinogenesis occurs at each of many hierarchical levels. Even at the genetic level, tumor cells accumulate mutations in multiple genes during formation of most cancer types. Cancer is also the outcome of altered mechanisms occurring at other levels involving RNA, proteins, intracellular pathways, intercellular interactions, tissues, organs, etc. Since events occurring at one hierarchical level feed into and modify mechanisms at other levels, cancer development is a dynamic process that is more complicated than a simple summation of the parts. An important consideration is that some cancers may originate from stem cells.

Thus, for cancer patients who face immunosuppressive therapy who have no readily matched donor, doctors have used “autologous” transplants: the cancer patient's bone marrow is removed, frozen, and stored until therapy is complete. Then the cells are thawed and re-infused into the patient.

Collection of stem cell products (SC products), a term which includes both true stem cells and committed progenitor cells (i.e., CD 34.sup.+cells are included), whether from bone marrow or peripheral blood, can be stored for future use, one of the most significant of which is transplantation to enhance hematologic recovery following an immunosuppressive procedure such as chemotherapy.

In the prior art, there is one significant drawback to the use of this very beneficial reinfusion procedure for treating a cancer patient. Breast cancer is thought to be systemic at the time of diagnosis. When SC products are obtained from the cancer patient, a significant number of tumor cells may also be collected, thereby contaminating the SC product.

Subsequently, when the SC product is reinfused into the cancer patient, the tumor cells are also reintroduced, increasing or re-introducing tumor cells into the patient's blood stream. While circulating tumor cells have not been directly linked to the relapse of a particular cancer, in the case of lymphoma, for example, reinfused cells have been traced to sites of disease relapse. In cases involving adenocarcinoma, it has been estimated that for a 50 kilogram adult, approximately 150,000 tumor cells can be reinfused during single stem cell transplantation. Moreover, it has been shown that the tumor cells present in the SC product are viable and capable of in vitro clonogenic growth, thus suggesting that they could indeed contribute to post-reinfusion relapse. Ovarian cancer cells, testicular cancer cells, breast cancer cells, multiple myeloma cells, non-Hodgkin's lymphoma cells, chronic myelogenous leukemia cells, chronic lymphocytic leukemia cells, acute myeloid leukemia cells, and acute lymphocytic leukemia cells are known to be transplantable.

The extent of tumor cell contamination of SC products appears to vary greatly from patient to patient, and values within the range of 11 to 78 percent have been recorded. Therefore, as the reinfusion of circulating tumor cells may well circumvent the benefits provided by aggressive chemotherapy followed by stem cell transplantation. See U.S. patent application, Publication No. 2001 0000204 A1, of Castino et al., published Apr. 12, 2001. Hereinafter referred to as “Castino et al.,” which is hereby incorporated by reference.

Methods currently used to separate the valuable stem cells from the undesired tumor cell-contaminated product rely on positive or negative selection techniques. Positive selection assays identify stem cells and progenitor cells that express markers for the CD34 antigen and remove them from the blood or bone marrow product contaminated with tumor cells. These methods are very labor intensive, reduce the number of useable stem cells and require the use of specialized equipment, thus greatly increasing the cost of patient care and severely limiting the use of SC products in transplantation procedures. An alternative to positive selection for removal of tumor cells from blood was provided by Gudemann et al., in an article entitled Intraoperative Autotransfusion In Urologic Cancer Surgery By Using Membrane Filters, XXIII.sup.rd Congress of the ISBT, abstracts in Vox Sang., (67 (S2), 22.), which is incorporated by reference, who described filtration with special leukocyte depletion membrane filters (which work by adsorbing charged particles) to remove urologic tumor cells from autologous blood during an intraoperative mechanical autotransfusion (IAT) procedure. A disadvantage of the membrane filters used by Gudemann et al is that they do not selectively retain tumor cells. White blood cells, including stem cells, are also retained. Thus, tumor cells are not removed from stem cells. The work of Miller et al also teaches that standard blood transfusion filters are ineffective at removing tumor cells from autologous blood.

On another front, in an attempt to improve the efficacy of stem cell therapy, scientists have exposed the cancer patients' extracted stem cells to modification in culture medium in the hope of “programming” them, such as to enhance their cancer fighting capability, before transfusing them into the patient. However, such studies have been unsuccessful in demonstrating a superiority of programmed stem cells versus native stem cells clinically.

Accordingly, there is a need to improve the benefits of stem cell transfusion which would ultimately result in increased survival rates, while at the same time providing a low-cost, clinically effective method for treating cancer patients with stem cell products.

### 3. Applicants' Hypotheses

The following hypotheses is made with regard to this invention. Each hypothesis may or may not relate to the other hypotheses. The efficacy of the invention in practice is not bound by the correctness of the hypotheses.

As a first hypothesis, applicants believe that many cancers are systemic in nature at the time of diagnosis and widely distributed throughout the body. That is, applicants believe that by the time a patient has been diagnosed with many types of cancer, e.g., breast cancer, there can already exist cancer cells in other parts of the patient besides the perceived affected area. In fact, the cancer may have already spread or migrated throughout the patient's body, for example, as the cancer cells are being carried by the patient's circulating blood or lymphoid system. This hypothesis accounts for contamination of stem cells, by cancer cells, collected from the patient, which may cause relapse after transplantation.

As a second hypothesis, applicants believe that in some instances, malignant or pre-malignant cells are routinely generated by the human body. However, the human does not develop cancer because his normal cells “self-regulate” the body by monitoring and eliminating the malignant or pre-malignant cells before they proliferate uncontrollably and give rise to cancer. This is termed “immune surveillance”. Applicants further postulate that in some cancer patients, their previously healthy cells become diseased because their diseased cells have partially or completely lost the ability to “self-regulate” due to old age, and/or environmental assaults (exposure to radiation, carcinogens, or stress, etc.); and/or other factors as yet unknown. Thus, transplantation of such already diseased (defective) cells harvested from these patients may not be helpful.

As a third hypothesis, applicants believe that certain diseases arise due to the loss of one or more functions of a healthy cells, due to old age, and/or environmental assaults (exposure to radiation, carcinogens, or stress, etc.); and/or other factors as yet unknown. Such loss may result in the failure to self-regulate, or to generally sustain normal functioning of the body. For example, the cell loses the ability to produce an enzyme or chemical necessary for the body's proper functioning. Thus, such a loss may result in Alzheimer disease, Parkinson disease, etc.

As a fourth hypothesis, the applicants believe that the programmed stem cells collected from cancer patients have not shown observable advantage over unprogrammed stem cells from the same patient, because both the programmed and unprogrammed cells are already diseased and thus damaged. That is, both the programmed and unprogrammed cells have lost their cancer fighting ability (or their optimal cancer fighting ability as compared to healthy cells), and the “programming” cannot restore the normal function to the already diseased cells (which have irretrievably lost their function) necessary to fight cancer. Again, this hypothesis can be generalized to any disease, besides cancer, wherein a healthy cell will be more readily programmed that a diseased cell (which may be partially or completely unresponsive to programming).

In summary, based on the above hypotheses, even though the present application uses cancer as an example of a disease for treatment under the invention, it is understood that other diseases (which result from the partial or complete loss of one or more abilities of a cell over time; or a systemic disease; or immunodiseases such as cancer) will similarly benefit from the present invention. Cancer is used herein merely for the convenience of illustration and discussion. The methods of the present invention can also be used to supplant immune cells to patients undergoing immunosuppressive treatments, such as chemotherapy, radiation therapy, or those who have been exposed to factors which deplete their bodies of immune cells.

### 4. The Invention

Thus, the present invention provides the following method:

(1) healthy cells are harvested from a person in the “pre-disease” stage. The term “pre-disease” indicates the state in which the patient is healthy, or before the patient has developed, manifested, or been diagnosed with a disease,

(2) the pre-diseased cells are preserved and stored, e.g., by cryopreservation;

(3) in the event that the person later develops, manifests, or been diagnosed with a disease (“post-disease” state), he is infused with the above previously preserved pre-disease cells. The term “post-disease” denotes the state at or after which the person develops, has developed, manifests, has manifested, has been diagnosed with a disease, or his disease has become detectable or been detected.

Another aspect of the invention is:

harvesting healthy cells from a person in a “pre-disease” stage, and preserving the healthy cells for the person so that the healthy cells can be later infused into the person for a medical treatment.

It should be noted that the above discussion over “pre-disease” state (versus “post-disease” state) covers the absolute term of “healthy/no disease” (versus “not healthy/diseased”) and a relative term of a gradation in the disease progression (“healthier than” or “less diseased” than post-disease state). Since “pre-disease” can be defined by a time prior to a person being diagnosed with a disease, he could be healthy in an absolute term or he might already have the disease but only that it has not manifested itself, been diagnosed or detected. Even in the latter scenario, for such a “pre-disease” state, it is possible that the disease may not be so widespread such that it has reached the cells collected; or even if the cells collected are diseased, they may be less aggressive or are of a healthier grade due to the early stage of their development, or the cells still retain some functioning necessary to combat the disease. Thus, the term “healthy” cells covers both the absolute term that the cells are healthy, and the term that, relatively speaking, these collected cells are healthier than what the patient (in his “post-disease” state) currently have in his body.

### 5. Advantages of the Present Invention

The present invention has the following advantages:

(1) Since the method uses autologous transplant, there is no need to expand time, money and energy in “cleansing” the SC or donor marrow of potentially dangerous mature T cells (thought to be important for the development of graft-versus-host-disease. This is especially advantageous because such cleansing process may introduce harmful impurities into the bone marrow. Examples of cleansing process are: chemicals or monoclonal antibody (OKT3) that specifically recognize and eliminate mature T cells.

In the case of patients with other diseases, the healthy harvested cells will not contain the disease vectors, such as in the case of AIDS patients, the pre-disease cells will not contain the AIDS virus;

(2) Since the method uses autologous transplant, there is no need to laboriously locate SC or bone marrow from other donor and to conduct tests to ensure that the SC or bone marrow matches that of the recipient. Further, there is no fear that matching SC or bone marrow may not be found or that crucial time is lost to test and locate such matching SC or bone marrow such that the recipient may be in mortal danger or be deceased by the time a match is found;

(3) Since the method uses autologous transplant, there is no fear of graft-versus-host disease;

or immune rejection.

(4) Since the method uses autologous transplant, there is no fear of transplant transmitted disease (e.g. HIV, CMV, hepatitis, syphilis etc.)

(5) Of particular importance is the fact that these autologous transplants are harvested prior to the development of disease (pre-disease stage) as compared to the post-disease stage. The pre-disease state autologous transplant has the following advantages over the post-disease state autologous transplant:

(I) the previously harvested pre-disease cells will be younger—Among the possible advantages associated with youth are: the cells will likely to be more resilient, more versatile, and would retain normal (or relative more normal) activities and a full range of (or broader range of) activities, and thus more well-equipped and more vigorous in combating a disease, as compared to the post-disease cells. Further, due to advance in age (e.g., in old age), certain population of cells may be depleted or no longer be available for harvesting at a later stage in a human's life. Also, certain functions or genes in older cells may be turned off, down-regulated, or lost, due to the natural aging process, aged related deterioration, mutation, or accumulated “wear-and-tear”, or environmental assaults over the years, etc. Further, older cells (post-disease versus pre-disease cells) may contain more mutations, defects, due to age or mistakes in the replication process, or environment assaults.

(II) the previously harvested (pre-disease) cells will be healthy cells (or healthier than the current cells existing in the patient) and can be cloned or programmed, or more readily programmable than the post-disease cells.

(III) the pre-disease population of the harvested cells will be healthy or will contain more healthier (or less diseased) cells than the post-disease population of cells, and thus the population of the pre-disease harvested cells will not be contaminated or be less contaminated by diseased cells which may be re-introduced into the patient and potentially cause a relapse.

For example, in the case of cancer treatment, the present invention has the following advantages over the prior art described above: the infused cells will not be contaminated with cancer cells (or will be less contaminated with cancer cells if the collection occurred after the disease has taken hold but before its diagnosis) as compared to the cells collected from a patient who has already developed cancer. Therefore, no laborious, time-consuming, inefficient methods (that may even inadvertently introduce undesirable chemicals) assays and screenings are required to cleanse the harvested cell population to remove cancer cells from the pre-disease harvested cells. Further, the present invention eliminates or reduces the possibility of causing a relapse through infusion of cancerous cells.

(IV) the population of harvested cells may be more “well-rounded” or more normal/healthy in that a full range of normally occurring cells will be present relative to diseased cell population. For example, the peripheral blood SC collected from an AIDS patient will be deficient in T cells which are decimated by the AIDS virus; but found in the previously healthy population of cells.

(V) Furthermore, hematopoietic stem and progenitor cells can potentially be multiplied in culture, before or after cryopreservation, thus expanding the number of stem cells available for therapy. Thus, the population of healthy cells may be increased by cloning and infused into the patient to greatly boost his immunodefense in the number of cells available and that the cells are healthy.

(VI) Furthermore, processed hematopoietic stem cells may undergo immuno-modulation or cellular adaptation inherent in the processing and cryopreservation technique.

### 5. An Embodiment of the Invention

Thus, in one embodiment of the invention: the stem cells of a child or an adult (“person”), while the child or adult is in a pre-disease state, are harvested and then preserved (such as cryopreservation). The harvesting (collection) process can be achieved using apheresis. There may be a need to infuse cell growth factors such as Granulocyte Colony Stimulating Factor 3-6 days prior to the collection. To preserve the stem cells collected for future used, cryopreservation can be used.

Later (and this may be years later), should the same person develops cancer, an immunodisease, infectious disease or undergoes therapy or is exposed to conditions which causes immunosuppression or infection or depletion of his immune cells, then the preserved stem cells or bone marrow are infused into the person to combat the disease. This can be achieved by intravenous infusion of the stem cell products. Similarly, the treatment protocol, and the criteria for determining the progress of the person and for adjusting the amount/dosage of cells to be infused can be achieved using standard transplantation practice.

Of course, the amount of stem cells collected should be sufficient for a major transplantation. If needed to, multiple collections should be done at an appropriate interval between collections (typically 90 days apart). However, as medicine advances, the preserved cells can be cloned and made to multiply or differentiate into the desired cell types before infusion into the person. If the person is deficient in certain subpopulation of cells, the subpopulation of cells from the preserved or cloned cells may be selected for in the future, and infused into the person. Furthermore, the harvested or cloned cells can be programmed by growing them in vitro with the person's diseased cells or tissues, or under stimulation by desired chemicals or cytokines before selecting for the desirable programmed cell and infusing them into the person. All these are conceivably achievable in the future.

In this embodiment, stem cells and bone marrow cells are chosen because they are versatile and because of their known use in cancer and immunodisease treatments and known methods for harvesting, preserving, cloning them. Their use in such treatments may be employed in this invention. The following describes this embodiment in further details.

### 5(a) Cancer Treatment

For ease of discussion, the following use breast cancer as a non-limiting example of cancer. The incident of breast cancer is the second highest, after lung cancer, in Caucasian women. Breast cancer is a difficult disease to treat. Patients undergoing chemotherapy, radiotherapy, or immunosuppressive therapy, generally lose immune cells. In the present invention, the patient's immune cells are replenished by her previously harvested pre-disease SC. Further, chemotherapy and radiotherapy destroy rapidly dividing cells which include cells found in bone marrow, the gastrointestinal tract (GI), and hair follicles. Thus, there is a threshold to the amount of chemical or radiation administered to the patient. The GI is more tolerant of higher dose of chemotherapy and radiotherapy than the bone marrow. Thus, with the stem cells replacement of this invention, a higher and more effective (aggressive) dose of chemotherapy or radiation may be administered to the patient to more aggressively eliminate the cancer cells.

### 5(b) Methods For SC Collection, Selection, Preservation and Infusion

Conventional methods for collecting, cryopreserving, thawing, screening for and quantifying stem cells, and selecting for subpopulations of the stem cells, may be used.

Apheresis collection process from peripheral blood is the most common for collecting stem cells today. Hematopoietic cells can be isolated from suitable animal or human tissues including, for example, peripheral blood and bone marrow. Mononuclear cells, for example peripheral blood mononuclear cells (PBMCs) may be further isolated by methods such as density-gradient centrifugation. Sufficient quantity should be collected. If needed to, multiple collections should be considered to ensure enough doses for most demanding transplantation, typically about one (1) billion cells. The stem cells may be preserved by cryopreservation and later thawed for use, using standard transfusion procedures.

In an embodiment of the invention, all the stem cells collected can be cryogenically preserved, and used for hematopoietic reconstitution after thawing, in order to avoid cell losses associated with cell separation procedures. However, it is envisioned that cell separation procedures can be used if desired.

In one embodiment of the present invention for the primitive cell population to be further subdivided into isolated subpopulations of cells that are characterized by specific cell surface markers. The methods of the present invention may further include the separation of cell subpopulations by methods such as high-speed cell sorting, typically coupled with flow cytometry.

### 5(c) Infusion and Transplantation

Conventional standard transfusion methods (e.g. intravenous infusion) may be used for infusing the stem cells. Standard protocols for chemotherapy may be used followed by stem cells infusion for bone marrow reconstitution.

### 5(d) Confirmation That the Transplant is Working

Normal conventional practice should be observed to monitor the progress of the patient undergoing transplantation. However, the applicants' method should reduce the amount of potential complications resulting from immune rejection, graft-versus-host-diseases, the duration of engraftment and infectious complications. The patient would benefit significantly because, if engraftment and reconstitution of the hematopoietic system does not occur after transplantation, the physician can rapidly detect this rejection and proceed with a second transplant

This application provides a long-lasting, efficient, and effective means for identifying biological materials, such as tissue, blood, and cells (the foregoing are herein collectively referred to as “biological material”). Non-limiting examples of such biological materials are: peripheral blood, stem cells, organs, and bone marrow cells. These biological materials can be collected or obtained from any animal (“donor”) which may be dead or alive. For example, biological material may be harvested from a brain dead person in a case of an organ transplant or a healthy person in a case of a blood transfusion for later transplant or infusion into another person (“transplant recipient”). Alternatively, a person may have his or her biological material harvested and preserved for his or her own later use. For ease of discussion, this application uses a human as a non-limiting example of a donor.

One embodiment of the invention is particularly useful for identifying a biological material which is potentially subject to transit or storage. Examples of such a biological material are blood which is stored and transported in blood unit (generally in the form of a plastic bag); tissues (such as pancreas) which are stored and transported in thermally insulated containers (generally in the form of a plastic Styrofoam insulated box).

Currently, a blood unit (plastic bag) is usually provided with an outside pocket into which paperwork containing identifying information regarding the blood contained therein. Alternatively, a barcode label is attached to the outside of the bag.

The information in the paperwork, label, or note is generally limited to identification data such as unit number. The current method is deficient and defective in that the paperwork or label is often lost, misplaced, destroyed, mixed up with other containers in transit or storage, or the writing becomes illegible over time, or due to exposure to liquid or changes in the environment, for example when the biological material is frozen.

In the case in which a biological material is transplanted into a recipient, if a mistake is made due to the mixed up of the paperwork or label or a misreading of an unclear writing, a potentially dangerous situation may arise, such as graft-versus-host disease due to the incompatibility of the biological material to the transplant recipient.

Further, currently, there is no independent convenient confirmation of the information on the paperwork or the label, or a backup in case the paperwork or label should be lost, destroyed, misplaced or become illegible.

To overcome the above deficiencies and to improve on the current method, the present invention presents the following:

The first embodiment of the invention provides a method for identifying a biological material based on its major histocompatibility complex (“MHC”) type. There are two main classes of MHC genes, Class I and Class II. The phrase “human leukocyte antigen” (“HLA”) refers to the MHC of humans which are immune response genes that encode cell surface glycoproteins that regulate interactions among cells of the immune system. These genes are involved in graft-versus-host rejection. The phrase “MHC restriction” refers to the recognition of peptides by T cells in the context of particular allelic forms of MHC molecules as described in Fundamental Immunology, 4th Ed., Paul (ed.) 1999.

Thus, in one embodiment of the invention, the biological material is identified by its HLA type. Such identification is particularly important if the biological material is to be later transplanted into the original donor of the biological material or another recipient. This is because biological material from a donor (“allogeneic” donor) must carry self-markers (MHC or HLA) that closely match those of the recipient. This match prevents the transplant from being rejected, but also to fend off a life-threatening situation known as graft-versus-host disease. In graft-versus-host disease, mature T cells from the donor attack and destroy the tissue of the recipient.

The second embodiment of the invention provides a method for identifying the biological material based on genetic materials in the biological material which serves as a unique set of “genetic fingerprint” which positively identifies the biological material as to its donor, versus a non-donor.

The third embodiment of the invention provides a method for identifying the biological material based on both its HLA type and genetic fingerprint—thus providing a double-confirmation, e.g., as to the donor of the biological material. This embodiment is useful in the case in which the biological material is harvested from the donor, stored, and intended for a later use.

The above embodiments of the invention may be carried out as follows:

The HLA gene sequence and/or genetic fingerprint of a biological material can be spotted on a solid substrate which can be an agarose, acrylamide, or polystyrene bead; a nylon or nitrocellulose membrane (for use in, e.g., dot or slot blot assays); a glass or plastic polymer; a silicon or silicon-glass (e.g., a microchip); or gold (e.g., gold plates).

In one embodiment of the invention, the solid substrate is a plastic microchip containing arrays of oligonucleotides (i.e., various binding members to be used to bind amplification products) which is the HLA and/or genetic fingerprint of the donor of the biological material. Generally, the HLA and/or genetic fingerprint are in the form of single-stranded DNA.

For ease of discussion, the following uses microchip as a non-limiting example of a substrate or array on which the identifying HLA and/or genetic fingerprint may be adhered.

Containers

Currently, containers for biological samples are available, such as plastic bag unit for transporting blood. Such plastic bags are usually provided with an outside pocket for inserting paperwork (e.g., the cryo bag commercially available from Baxter Company, State of California). Instead of or in addition to the usual paperwork, the microchip of the present invention can be placed in the pocket and the pocket is preferably sealed to avoid the loss of the microchip. Further, in another embodiment of the invention, the microchip may have an adhesive backing to adhere it to the bag within the pocket. The microchip may be attached or detached manually from the bag; or it may be permanently attached to the bag and is removed by cutting around the surrounding plastic. In yet another embodiment, the microchip may be placed in a sealed plastic bag to avoid damage, and then inserted into the container of the biological material or inserted into the outer pocket of the container in which the biological material is stored.

Hybridization

When the stored biological material is ready for transplant or when one wishes to identify the stored biological material, the microchip is retrieved and complementary DNA to the DNA of the biological material is made and hybridized to the microchip under suitable hybridization conditions. The hybridization to the site in the microchip confirms the presence of the particular HLA gene and/or genetic fingerprint.

If HLA typing is required, then the DNA can be that of the specifically desirable HLA type. Hybridization between the DNA on the microchip and the complementary DNA of the desired HLA would confirm HLA matching between the biological material and the specific HLA type. The specific HLA type can be that of the donor in the case in which one wishes to confirm the biological material has HLA type consistent with that of the donor or the recipient in the case in which one wishes to confirm the biological material has HLA type suitable for transplant into the recipient. If one wishes to confirm that the biological material is from the donor, the donor's DNA fingerprint can be derived from a biological material freshly removed from the donor, and hybridization of the new complementary DNA with the DNA on the microchip would confirm the identity of the donor. For double confirmation, microchip containing both HLA and genetic fingerprint may be used, and hybridization of both to the newly derived DNA from biological material freshly collected from the donor would confirm their identical source (i.e., from the same donor).

U.S. Pat. No. 6,183,968 (to Bandman et al.), which is hereby incorporated by reference, discloses polynucleotide probes that can be used as hybridizable array elements in a microarray, each of the polynucleotide probes having at least a portion of a gene which encodes a protein associated with cell proliferation or a receptor. The method of this patent may be modified as follows: instead of having each of the polynucleotide probes having at least a portion of a gene which encodes a protein associated with cell proliferation or a receptor; the polynucleotide of the present invention may have each of the polynucleotide probes having at least a portion of a gene which encodes a MHC specific to the donor and/or polynucleotide probes for genetic fingerprint of the donor.

Nucleic acid hybridization and wash conditions may be chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls. Optimal hybridization conditions will depend on the length (e.g., oligomer versus polynucleotide greater than 200 bases) and type (e.g., RNA, DNA, PNA) of labeled probe and immobilized polynucleotide or oligonucleotide.

Another aspect of the invention presents a kit comprising a container containing a biological material, and a substrate having the HLA and/or genetic fingerprint of the donor, said substrate and HLA and/or genetic fingerprint being suitable for a nucleotide hybridization test. Another aspect of the invention presents a substrate having the HLA and/or genetic fingerprint of a specific individual. Another aspect of the invention presents a method for identifying the HLA and/or genetic fingerprint of a specific individual and placing those nucleotide sequences of the HLA and/or genetic fingerprint onto a solid substrate.

A further aspect of the invention comprises placing the solid substrate containing nucleotide sequences of HLA and/or genetic fingerprint of a specific individual in a container of said individual's biological material.

A further aspect of the invention comprises identifying a specific individual's biological material by a solid substrate containing nucleotide sequences of HLA and/or genetic fingerprint of the individual. Said substrate being selected from the group consisting of: agarose, acrylamide, or polystyrene bead; a nylon or nitrocellulose membrane (for use in, e.g., dot or slot blot assays); a glass or plastic polymer; a silicon or silicon-glass (e.g., a microchip); or gold; or a plastic microchip.

Although the foregoing invention has been described in some detail by way of illustration and example for purposes of clarity and understanding, the above are by way of example, and are not meant to be limiting. It will be obvious that various modifications and changes which are within the skill of those skilled in the art are considered to fall within the scope of the appended claims.

What is claimed is:

1. A method of analyzing the cellular integrity of cells, the method comprising:
harvesting pre-disease cells from an individual; and
preserving the integrity of the pre-disease cells so that the pre-disease cells can provide a benchmark for comparing later cells collected from the same individual for measuring the progression of the later cells.

2. The method according to claim 1, where the later cells are initial-disease cells.

3. The method according to claim 1, where the later cells are post-disease cells.

4. A method for treating infectious disease, neutropenia, and aplastic anemia, in an individual with autologous stem cells, the method, comprising:
collecting autologous stem cells from an individual while the individual is in a healthy state; and
preserving the autologous stem cells so that the substantially similar autologous stem cells can be transfused into the individual for treating the individual when the individual is diagnosed with having infectious agents such as bacteria, virus, fungi, parasite, prion protein, and the like.

5. The method of claim 4, wherein the autologous stem cell units include hematopoietic and non-hematopoietic stem cells.

6. The method of claim 4, wherein the autologous stem cells are collected from peripheral blood of the individual.

7. The method of claim 4, wherein the autologous stem cells are obtained from bone marrow of the individual.

8. A method for treating certain cancers such as breast cancer, in an individual with autologous stem cells, the method comprising:
collecting autologous stem cells from an individual while the individual is in a healthy state; and
preserving the autologous stem cells so that the substantially similar autologous stem cells can be transfused into the individual for treating the individual when the individual is diagnosed with having breast cancer or other malignancies.

9. The method of claim 9, wherein the autologous stem cell units include hematopoietic and non-hematopoietic stem cells.

10. The method of claim 8, wherein the autologous stem cells are collected from peripheral blood of the individual.

11. The method of claim 8, wherein the autologous stem cells are obtained from bone marrow of the individual.

12. A method of measuring a progression of cells from an individual, the method comprising:
harvesting a first set of cells from an individual;
preserving the first set of cells to maintain the cellular integrity of the first set of cells;
harvesting a second set of cells from the same individual at a later time than the harvesting of the first set of cells; and
comparing the difference between a first set of cells from the second set of cells to measure the progression of cells in the individual from the first set of cells to the second set of cells.

13. A method of diagnosing a disease of an individual, the method comprising:
comparing differences between pre-disease cells of the individual and cells harvested from the same individual with a disease at a later time; and
treating the individual with the disease based on the differences between the pre-disease cells and cells harvested from the same individual at the later time.

14. The method according to claim 13, including:
harvesting the pre-disease cells from the individual; and
preserving the first set of cells to maintain the cellular integrity of the first set of cells.

15. The method according to claim 13, where the comparing includes molecular differences in DNA between the pre-disease cells and cells harvested from the same individual at the later time.

16. The method according to claim 13, where the comparing includes molecular differences in RNA between the pre-disease cells and cells harvested from the same individual at the later time.

17. The method according to claim 13, where the comparing includes molecular differences in protein expression between the pre-disease cells and cells harvested from the same individual at the later time.

18. The method according to claim 13, where the comparing includes molecular differences in gene expression between the pre-disease cells and cells harvested from the same individual at the later time.

19. The method according to claim 18, including:
analyzing the changes in the gene expression between the pre-disease cells and cells harvested from the same individual at the later time to detect early stage of the disease.

20. A system capable of measuring the cellular integrity of cells from an individual, the system comprising:
means for harvesting cells from an individual;
means for preserving the cells to maintain the cellular integrity of the cells; and
means for comparing the cells harvested at different times from the individual to measure a progression of the cells harvested at a later time from the individual.

21. A system capable of identifying biological material, the system comprising:
means for containing genetic information of the biological material on a microchip array, where the genetic information includes HLA information.

22. The system according to claim 21, including:
means for confirming the biological material in the microchip array.

23. The method of claim 21, wherein the genetic information includes nucleotide sequences of HLA and/or genetic fingerprint of the biological material.

24. The method of claim 21, wherein the microchip array is selected from a group consisting of agarose, acrylamide, polystyrene bead, nylon, nitrocellulose membrane, glass, plastic polymer; silicon, silicon-glass, gold, and plastic microchip.

25. A kit comprising:
a container having a biological material, and
a substrate having the HLA and/or genetic fingerprint of the donor, said substrate and HLA and/or genetic fingerprint being capable of a nucleotide hybridization test.

26. A method of destroying tumor cells, the method comprising:
collecting autologous stem cells from an individual prior to diseased state; and
using the autologous stem cells for immuno-surveillance to destroy tumor cells and fight infections in the individual, whereby the autologous stem cells collected prior to diseased state are more readily available for infusion than stem cells collected during diseased state and the autologous stem cells collected prior to the diseased state are better able to fight cancer cells in the individual than stem cells collected from the individual at a diseased state.
